(19) United States
(12) Patent Application Publication
(10) Pub. No.: US 2005/0149108 A1
(43) Pub. Date: Jul. 7, 2005

(54) Implant delivery and detachment system and method

(75) Inventor: Brian J. Cox, Laguna Niguel, CA (US)

Correspondence Address: O'MELVENY & MEYERS, 114 PACIFICA, SUITE 100, IRVINE, CA, 92618, US

(73) Assignee: Microvention, Inc.

(21) Appl. No.: 10/739,646

(22) Filed: Dec. 17, 2003

(51) Int. Cl. A61M 29/00

(52) U.S. Cl. 606/200

(57) ABSTRACT

The present invention provides for implant device delivery apparatuses and related methods of use. An apparatus of the present invention may include a pusher member selectively engaged to an implant device. The implant device may be, e.g., a coil. A coupling section of the apparatus allows the implant device to be secured to the pusher member via an application of energy. When it is desired to disengage the implant device, such as after locating the implant device at a target cavity site, the application of energy is ceased and the implant device may be disengaged from the pusher member.

### FIELD OF THE INVENTION

The present invention relates to systems and methods for the deployment and release of medical implant devices within the body of a patient.

### BACKGROUND

The embolization of blood vessels and other luminal organs is desired in a number of clinical situations. For example, vascular embolization has been used to control vascular bleeding, to occlude the blood supply to tumors, and to occlude vascular aneurysms, particularly intracranial aneurysms. In recent years, vascular embolization for the treatment of aneurysms has received much attention. Several different treatment modalities have been employed in the prior art. U.S. Pat. No. 4,819,637 to Dormandy, Jr. et al., for example, describes a vascular embolization system that employs a detachable balloon delivered to the aneurysm site by an intravascular catheter. The balloon is carried into the aneurysm at the tip of the catheter, and it is inflated inside the aneurysm with a solidifying fluid, such as a polymerizable resin or gel, to occlude the aneurysm. The balloon is then detached from the catheter by gentle traction on the catheter. While the balloon-type embolization device can provide an effective occlusion of many types of aneurysms, it is difficult to retrieve or move after the solidifying fluid sets, and it is difficult to visualize unless it is filled with a contrast material. Furthermore, there are risks of balloon rupture during inflation and of premature detachment of the balloon from the catheter.

Another approach is the direct injection of a liquid polymer embolic agent into the vascular site to be occluded. One type of liquid polymer used in the direct injection technique is a rapidly polymerizing liquid, such as a cyanoacrylate resin and particularly isobutyl cyanoacrylate, which is delivered to the target site as a liquid, and then is polymerized in situ. Alternatively, a liquid polymer that is precipitated at the target site from a carrier solution has been used. An example of this type of embolic agent is a cellulose acetate polymer mixed with bismuth trioxide and dissolved in dimethyl sulfoxide (“DMSO”). Another type is ethylene glycol copolymer dissolved in DMSO. Upon contact with blood, the DMSO diffuses out, and the polymer precipitates out and rapidly hardens into an embolic mass that conforms to the shape of the aneurysm. Other examples of materials used in this “direct injection” method are disclosed in U.S. Pat. No. 4,551,132 to Pasztor et al., U.S. Pat. No. 4,795,741 to Leshchiner et al., U.S. Pat. No. 5,525,334 to Ito et al., and U.S. Pat. No. 5,580,568 to Greff et al.

The direct injection of liquid polymer embolic agents has, however, proven difficult in practice. For example, migration of the polymeric material from the aneurysm and into the adjacent blood vessel has presented a problem. In addition, visualization of the embolization material requires that a contrasting agent be mixed with it, and selecting embolization materials and contrasting agents that are mutually compatible may result in performance compromises that are less than optimal. Furthermore, precise control of the deployment of the polymeric embolization material is difficult, leading to the risk of improper placement and/or premature solidification of the material. Moreover, once the embolization material is deployed and solidified, it is difficult to move or retrieve.

Another approach that has shown promise is the use of thrombogenic microcoils. These microcoils may be made of a biocompatible metal alloy, typically platinum and tungsten, or a suitable polymer. If made of metal, the coil may be provided with DACRON fibers to increase thrombogenicity. The coil is deployed through a microcatheter to the vascular site. Examples of microcoils are disclosed in U.S. Pat. No. 4,994,069 to Ritchart et al., U.S. Pat. No. 5,133,731 to Butler et al., U.S. Pat. No. 5,226,911 to Chee et al., U.S. Pat. No. 5,312,415 to Palermo, U.S. Pat. No. 5,382,259 to Phelps et al., U.S. Pat. No. 5,382,260 to Dormandy, Jr. et al., U.S. Pat. No. 5,476,472 to Dormandy, Jr. et al., U.S. Pat. No. 5,578,074 to Mirigian, U.S. Pat. No. 5,582,619 to Ken, U.S. Pat. No. 5,624,461 to Mariant, U.S. Pat. No. 5,645,558 to Horton, U.S. Pat. No. 5,658,308 to Snyder, and U.S. Pat. No. 5,718,711 to Berenstein et al.

The microcoil approach has met with some success in treating small aneurysms with narrow necks, but the coil must be tightly packed into the aneurysm to avoid shifting that can lead to recanalization. Microcoils have been less successful in the treatment of larger aneurysms, especially those with relatively wide necks. A disadvantage of microcoils is that they are not easily retrievable and detached; if a coil migrates out of the aneurysm, a second procedure to retrieve it and/or move it back into place is necessary. Furthermore, complete packing of an aneurysm using microcoils can be difficult to achieve in practice.

A specific type of microcoil that has achieved a measure of success is the Guglielmi Detachable Coil (“GDC”). The GDC employs a platinum wire coil fixed to a stainless steel guidewire by a solder connection. After the coil is placed inside an aneurysm, an electrical current is applied to the guidewire, which heats sufficiently to melt the solder junction, thereby detaching the coil from the guidewire. The application of the current also creates a positive electrical charge on the coil, which attracts negatively-charged blood cells, platelets, and fibrinogen, thereby increasing the thrombogenicity of the coil. Several coils of different diameters and lengths can be packed into an aneurysm until the aneurysm is completely filled. The coils thus create and hold a thrombus within the aneurysm, inhibiting its displacement and its fragmentation.

The advantages of the GDC procedure are the ability to withdraw and relocate the coil during delivery, and the enhanced ability to promote the formation of a stable thrombus within the aneurysm. Nevertheless, as in conventional microcoil techniques, the successful use of the GDC procedure has been substantially limited to small aneurysms and those with narrow necks.

There has thus been a long-felt, but as yet unsatisfied need for an aneurysm treatment device and method that can substantially fill aneurysms of a large range of sizes, configurations, and neck widths with a thrombogenic medium with a minimal risk of inadvertent aneurysm rupture or blood vessel wall damage. There has been a further need for such a method and device that also allow for the precise locational deployment of the medium, while also minimizing the potential for migration away from the target location. In addition, a method and device meeting these criteria should also be relatively easy to use in a clinical setting. Furthermore, there has been an unmet need for an aneurysm treatment device and method that allows for improved detachment and deployment of an implant or coil from the guidewire or other positioning device.

### SUMMARY OF THE INVENTION

The present invention is directed to implant device delivery apparatuses and methods in which energy is applied to the apparatus to secure the implant device to a pusher member. After placement at a desired location, the implant device is delivered by discontinuing the application of energy to the apparatus, which results in the implant device disengaging from the pusher member.

In a first aspect of the present invention, an implant device delivery apparatus is provided. The apparatus includes an implant device, a pusher member with a proximal end and a distal end, and a coupling section configured to attach the implant device to the distal end of the pusher member. The coupling section may include a thermally responsive member and a coupler adopted to receive the thermally responsive member. The thermally responsive member is held by the coupler by an application of energy and is released from the coupler by discontinuing the application of energy. The coupler may be attached to or in operable connection with the implant device, and the thermally responsive member may be attached to or in operable connection with the pusher member.

The coupling section of this apparatus may also include a resistive heating element that is in physical contact with, in close proximity to, or otherwise in communication with the thermally responsive member. The resistive heating element may be located within a lumen of the pusher member. The coupling section of this apparatus may also include a spring member within the pusher member or on the distal end of the pusher member. When present, the spring member is designed to forcibly release the thermally responsive member from the coupler after the application of energy is stopped. The implant device of this apparatus may take any of a number of suitable forms, including without limitation a coil or an implant device having a hydrophilic element.

In a second aspect of the present invention, another implant device delivery apparatus is provided. The apparatus includes a pusher member, a coupling section, and an implant device, which may be, without limitation, a coil, a hydrophilic element, or a coil with a hydrophilic element. The coupling section is configured to couple the implant device to the distal end of the pusher member, and includes a thermally responsive member, a retaining sleeve surrounding the thermally responsive member, and a tether detachably engaged between the thermally responsive member and the retaining element. The implant device is coupled to the pusher member by an application of energy to the coupling section, and is released from the pusher member by discontinuing the application of energy.

The thermally responsive member of this apparatus has a first configuration in which the thermally responsive member engages the tether, and a second configuration in which the thermally responsive member disengages the tether. The thermally responsive member may be placed in the first configuration by an application of energy to the coupling section. The coupling section may further include a resistive heating element in contact with or otherwise in communication with the thermally responsive member that may be used to apply energy to the thermally responsive member.

In one embodiment of this apparatus, the tether is permanently attached to the implant device. In another embodiment of this apparatus, the tether has a first end permanently engaged between the retaining sleeve and the thermally responsive member and a second end releasably engaged between the retaining sleeve and the thermally responsive member. The second end of this tether may be released from between the retaining sleeve and the thermally responsive member by discontinuing the application of energy. Additionally, the coupling section of this apparatus may also incorporate an attachment point that may be disposed on the proximal end of implant device. The tether may be detachably engaged to the attachment point by the application of energy and released from the attachment point by discontinuing the application of energy.

In a third aspect of the present invention, a method for delivering an implant device using a pusher member is provided. The implant device is affixed to the distal end of the pusher member by applying energy to the pusher member. The implant device is preferably affixed to the pusher member prior to introducing the implant device into the body. Affixing the implant device may be performed using electrical energy. Additionally, the implant device may be a coil having a proximal end and a distal end, and affixing the implant device may be accomplished by coupling the proximal end of the coil to the distal end of the pusher member prior to applying energy to the pusher member.

The implant device is then introduced into a body having a target cavity. The implant device is advanced to the target cavity. Once placed as desired, the implant device is detached from the distal end of the pusher member by discontinuing the application of energy to the pusher member. The pusher member is then removed from the body after the implant device is detached from the pusher member.

These and other objects and features of the present invention will be appreciated upon consideration of the following drawings and detailed description.

### BRIEF DESCRIPTION OF THE FIGURES

FIG. 1 is a cross-sectional view of an implant device delivery apparatus of the present invention.

FIG. 2 is a cross-sectional view of an implant device delivery apparatus of the present invention that incorporates a shape memory material to engage and disengage an implant device from a pusher member.

FIG. 3 is a cross-sectional view of an implant device delivery apparatus of the present invention that incorporates a tether to engage and disengage an implant device from a pusher member.

FIG. 4 is a cross-sectional view of another implant device delivery apparatus of the present invention that incorporates a tether to engage and disengage an implant device from a pusher member.

FIG. 5 is a schematic representation of a step of a method for delivering an implant device using the present invention.

FIG. 6 is a schematic representation of another step of a method for delivering an implant device using the present invention.

### DETAILED DESCRIPTION

The present invention provides for an implant device delivery apparatus and related methods. The apparatus includes a pusher member and an implant device, such as, e.g., a stent, a vascular filter, a vaso-occlusive coil and the like, and is configured so that a distal end of the pusher member releasably engages a proximal end of the implant device. The section of the apparatus that includes the engagement point between the pusher member and the implant device may be described herein as a coupling section. The attachment of the implant device to the pusher member is affected or enhanced when a thermally responsive member of the coupling is heated or otherwise energized to result in an increase in temperature of a thermally responsive member in the coupling section. Uniquely, the attachment of the implant device to the pusher member is done prior to insertion and is maintained using an application of energy. Also contrary to known prior art devices, the apparatus releases the implant device when the application of energy is removed, turned off, or otherwise discontinued. This invention is useful for the delivery of various devices into the vasculature and other body organs for therapeutic and/or diagnostic purposes.

Turning to FIG. 1, an implant device delivery apparatus 100 of the present invention is illustrated. The apparatus 100 includes an elongate pusher member 102 configured for use in advancing an implant device 104 into and within the body of a patent and, specifically, into a target cavity site for implantation and delivery of the implant device 104. Potential target cavity sites include blood vessels and vascular sites, such as, e.g., aneurysms and fistula, heart openings and defects, such as, e.g., the left atrial appendage, and other luminal organs, such as, e.g., fallopian tubes.

As illustrated, the implant device 104 is an embolic coil. An embolic coil suitable for use as the implant device 104 may comprise a suitable length of wire formed into a helical microcoil. The coil may be formed from a biocompatible material including platinum, rhodium, palladium, rhenium, tungsten, gold, silver, tantalum, and various alloys of these metals, as well as various surgical grade stainless steels. Specific materials include the platinum/tungsten alloy known as Platinum 479 (92% Pt, 8% W, available from Sigmund Cohn, of Mount Vernon, N.Y.) and nickel/titanium alloys (such as the nickel/titanium alloy known as “nitinol”). Another material that may be advantageous for forming the coil is a bimetallic wire comprising a highly elastic metal with a highly radiopaque metal. Such a bimetallic wire would also be resistant to permanent deformation. An example of such a bimetallic wire is a product comprising a nitinol outer layer and an inner core of pure reference grade platinum, available from Sigmund Cohn, of Mount Vernon, N.Y., and Anomet Products, of Shrewsbury, Mass. In specific embodiments of coils usable as the implant device 104, wire diameters of about 0.0125 mm to about 0.150 mm may be used. Commonly-assigned U.S. Pat. No. 6,605,101 provides a further description of embolic coils suitable for use as the implant device 104, including coils with primary and secondary configurations wherein the secondary configuration minimizes the degree of undesired compaction of the coil after deployment. The disclosure of U.S. Pat. No. 6,605,101 is fully incorporated herein by reference.

In another embodiment, the implant device 104 is a continuous, filamentous extrusion of polymeric “transition material” that is initially in a soft, self-adherent, compliant state and, after insertion, forms a web-like mass of material that substantially fills the target cavity site and substantially conforms to the interior shape of the site after placement therein. A suitable continuous, filamentous extrusion of “transition material” usable for the implant device 104 is disclosed in commonly-assigned U.S. Pat. No. 6,015,424, which is fully incorporated by reference herein.

The implant device 104 may also take the form of an expansible, hydrophilic embolizing elements connected to a flexible filamentous carrier, or a hydrophilic element disposed at the distal end of the carrier. The carrier may be a suitable length of very thin, highly flexible filament of a biocompatible alloy or metal, including the materials previously identified for use in forming a coil. The hydrophilic embolizing elements or element may be manufactured from a biocompatible, macroporous, hydrophilic hydrogel foam material. An exemplary hydrogel is an environmentally-sensitive porous hydrogel polymer such as the type disclosed in commonly-assigned U.S. Patent Application Publication No. US 2002/0176880 A1, which is fully incorporated by reference herein. Other suitable hydrophilic materials are disclosed in U.S. Pat. No. 5,570,585, which is also fully incorporated by reference herein. Preferably, the hydrophilic hydrogel material expands after being placed within the target cavity site due to, e.g., contact with blood or other bodily fluids. An example flexible carrier having hydrophilic embolizing elements that may be used as the implant device 104 is disclosed in commonly-assigned U.S. Pat. No. 6,238,403 and commonly-assigned U.S. Pat. No. 6,165,193, the disclosures of which are fully incorporated by reference herein.

Alternatively, the implant device 104 may be a stent, a vascular filter, or other device suitable for implantation within the target cavity site.

The pusher member 102 is preferably a long, thin, hollow, and highly flexible tube. The pusher member 102 has an axial passage or lumen 103 between its proximal and distal ends. In one embodiment, the pusher member 102 is formed from stainless steel. In other embodiments, the pusher member 102 may be formed from another biocompatible metal, a biocompatible plastic material, or any other suitable biocompatible material. In one embodiment, the pusher member 102 is made at least in part of a material that allows external visualization under various medical imaging methods, such as, e.g., x-ray, magnetic resonance imaging (“MRI”), or ultrasound. Furthermore, in an alternative embodiment the pusher member 102 is a substantially solid cylindrical member having an energy transmission or application component, such as, e.g., the resistive heat element 116 that is described herein, embedded within the pusher member 102.

In one implementation of the apparatus 100, the pusher member 102 is used to advance the implant device 104 through a tubular access device (not shown), such as, e.g., a cannula, a catheter, and the like, in order to place the implant device 104 in or near the target cavity site. In another embodiment, the pusher member 102 is used to position the implant device 104 without the use of an additional tubular access device.

The apparatus 100 has a coupling section 106 that defines the engagement point between the pusher member 102 and the implant device 104. The coupling section 106 includes a coupler 108 that is attached to the proximal end of the implant device 104. As shown in FIG. 1, at least the distal portion of the coupler 108 is attached to the interior of the implant device 104 via a bond or a weld 110, but any suitable attachment method or adhesive material may be used. The coupler 108 is preferably formed from any suitable biocompatible metallic or biocompatible plastic material. The proximal portion of the coupler 108 incorporates an opening 112 that is configured to receive at least a portion of a thermally responsive member 114 that is located at the distal end of the pusher member 102.

Accordingly, the coupling section 106 also includes a thermally responsive member 114. The thermally responsive member 114 preferably expands from an unexpanded state to an expanded state when energy is applied to the thermally responsive member 114, and returns to the unexpanded state after the application of energy is discontinued. In the embodiment shown in FIG. 1, energy is preferably applied to the thermally responsive member 114 using a resistive heating element 116. Here, the resistive heating element 116 is formed by twin electrical lead wires, specifically a positive electrical lead wire 116(a) and a negative electrical lead wire 116(b), that extend through the lumen 103 of the pusher member 102 and wrap around the proximal end of the thermally responsive member 114. In other embodiments of the apparatus 100, the resistive heating element 116 may be formed from any suitable energy transmission component that allows for externally applied energy to be delivered to the distal end of the pusher member 102 and ultimately to the proximal end of the thermally responsive member 114. Exemplary energy transmission components that may be incorporated into the apparatus 100 to apply energy to the thermally responsive member 114 include, e.g., electrical components, radio frequency (“RF”) components, ultrasound components, thermal energy components, and the like.

The proximal end of the thermally responsive member 114 is in operable connection with the resistive heating element 116, as shown, or alternatively placed in sufficiently close proximity to the resistive heating element 116 to allow thermal conduction from the resistive heating element 116 to the thermally responsive member 114. The distal end of thermally responsive member 114 engages with the opening 112 of the coupler 108 when it expands to its expanded state due to the application of energy from the resistive heating element 116.

The opening 112 of the coupler 108 preferably has a diameter that is only slightly or marginally larger than the diameter of the distal end of the thermally responsive member 114, as shown in FIG. 1, to allow the distal end of the thermally responsive member 114 to be inserted therein when the thermally responsive member 114 is in an unexpanded state. When energy, such as, e.g., an electrical current, is delivered through the pusher member 102 via the resistive heating element 116, the energy is transferred to the thermally responsive member 114, thereby heating the thermally responsive member 114. The thermally responsive member 114 expands due to thermal expansion when heat energy is applied to it via the resistive heating element 116. The thermally responsive member 114 is heated to a temperature sufficient to increase the diameter of the thermally responsive member 114, and specifically the distal end of the member 114, to at least substantially the same diameter as the opening 112 of the coupler 108. In one embodiment, the thermally responsive member 114 is formed from a material that expands to a sufficient diameter when the member 114 reaches a temperature of at least about 38° C. The thermally responsive member 114 is preferably made of a material with a relatively high coefficient of thermal expansion, such as, e.g., a material having a coefficient of thermal expansion of at least about 1.5×10⁻⁵ mm/mm/° C. The thermally responsive member 114 may, therefore, be made of a metal, a polymer, or a composite material so long as the material has a sufficiently high coefficient of thermal expansion.

The expansion of the distal end of the thermally responsive member 114 within the opening 112 of the coupler 108 substantially increases the frictional resistance to detachment of the implant device 104 from the pusher member 102. The frictional resistance is sufficiently high such that an axial force is required to overcome the frictional engagement and thereby detach the implant device 104 from the pusher member 102. In one preferred embodiment, the axial force required to detach the implant device 104 from the pusher member 102 when the thermally responsive member 114 is in its expanded state within the opening 112 of the coupler 108, i.e., when energy is being applied to the thermally responsive member 114, is at least about 45 grams (0.1 lbs). In one embodiment, energy is continually applied to the thermally responsive member 114 to maintain the member 114 in its expanded state and keep the implant device 104 attached to the pusher member 102. In another embodiment, energy is intermittently applied to the thermally responsive member 114 with a frequency that is sufficient to maintain the member 114 in the expanded state.

When it is desired to detach the implant device 104 from the pusher member 102, such as, e.g., after the implant device 104 has been placed at a desired target cavity site within a body, the energy delivered from the resistive heating element 116 through the pusher member 102 is stopped, such as via a switch used to open or close an electrical circuit. After the delivery of energy from the resistive heating element 116 is discontinued, the thermally responsive member 114 cools and returns to its unexpanded state. The cooling of the thermally responsive member 114 may be facilitated by local blood flow when the local blood flow temperature is less than the temperature of the thermally responsive member 114 in the heated, expanded state, or by the injection of a biocompatible fluid, such as, e.g., saline solution, through the pusher member 102 and to the thermally responsive member 114. When the thermally responsive member 114 returns to its unexpanded state, the opening 108 of the coupler 108 is no longer frictionally engaged to the distal end of the thermally responsive member 114, and the implant device 104 is released from the pusher member 102.

In one embodiment, a spring member 118 is optionally disposed on the distal end of the pusher member 102 or on the distal end of the thermally responsive member 114, as shown in FIG. 1. In embodiments of the apparatus 100 that include the spring member 118, the spring member 118 becomes compressed when the thermally responsive member 114 is placed within and engaged to the opening 112 of the coupler 108. Then, when the application of energy to the thermally responsive member 114 is discontinued, the thermally responsive member 114 returns to its unexpanded state and the spring member 118 decompresses and applies a disengagement force that aids in separating the implant device 104 from the pusher member 102.

In addition to the noted attributes, a median portion of the coupling section 106 (and the coupling sections of the other embodiments of the apparatuses of the present invention discussed herein) defines a space between the distal end of the pusher member 102 and the proximal end of the implant device 102. The space separating the distal end of the pusher member 102 and the proximal end of the implant device 102 imparts an increased degree of maneuverability to the apparatus 100. For example, the space between the pusher member 102 and the implant device 104 allows the implant device 104 to be maneuvered and placed at a greater range of angles relative to the pusher member 102 in comparison to an embodiment in which the distal end of the pusher member 102 and the proximal end of the implant device 104 are in direct connection when an apparatus is in the engaged state.

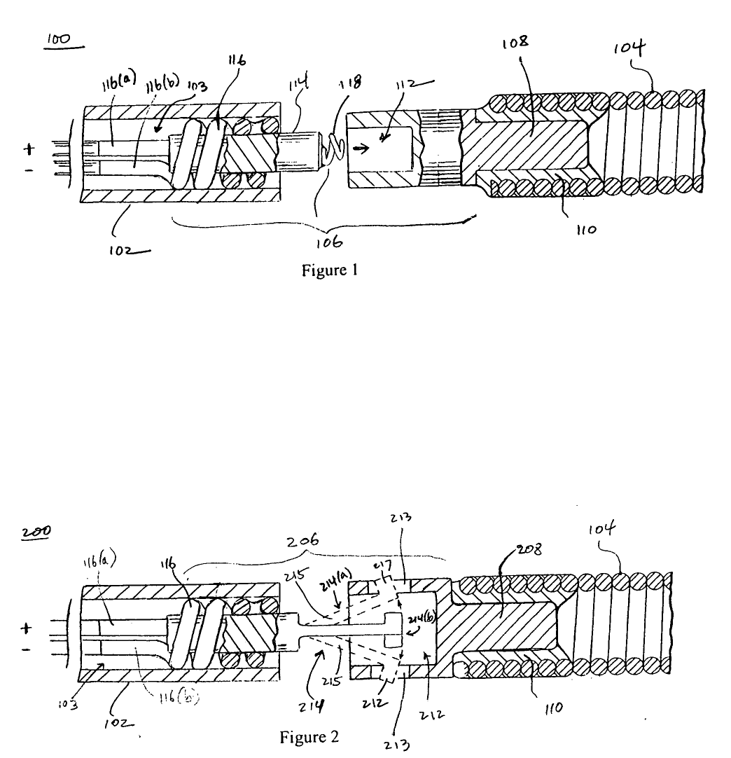

Turning to FIG. 2, another embodiment of the present invention, implant device delivery apparatus 200, is illustrated. Apparatus 200 shares several common elements with apparatus 100. For example, the same devices usable as the implant device 104 with apparatus 100 are also usable as the implant device 104 with apparatus 200. These include, e.g., an embolic microcoil/coil, a continuous filamentous extrusion of polymeric “transition material,” a flexible filamentous carrier having one or more expansible, hydrophilic embolizing elements, a flexible filamentous carrier having a hydrophilic element at its distal end, a stent, a vascular filter, and the like. These implant devices 104 have been previously described with respect to apparatus 100. As with the implant device 104, the same identification numbers are used to identify other elements/components of apparatus 100 that are also used in apparatus 200. Reference is made to the description of these elements in the description of apparatus 100 as that description also applies to these common elements in apparatus 200.

Apparatus 200 includes a coupling section 206 with a thermally responsive member 214 and a coupler 208. The thermally responsive member 214 is a mechanical structure capable of selectively engaging the coupler 208 in an interlocking fashion. The coupler 208 includes an opening 212 within which the thermally responsive member 214 may be situated while the thermally responsive member 214 is engaged to the coupler 208. As with apparatus 100, at least the distal portion the coupler 208 is affixed to the interior space of the proximal portion of the implant device 104 via, e.g., a bond or weld 110, or other suitable attachment or adhesive means.

The thermally responsive member 214 is formed at least in part of a shape memory material. Preferably, the shape memory material is processed to exhibit a two-way shape memory, rather than a typical one-way shape memory, in order to allow rapid engagement and disengagement with the coupler 208. Unlike one-way shape memory in which the shape memory material remembers only a high temperature shape, a shape memory material trained to have a two-way shape memory remembers both a high temperature shape and a low temperature shape. Two-way shape memory may be imparted to shape memory alloys such as nickel titanium (NiTi or nitinol) by several processes known in the art. Exemplary processes are described in Huang et al., “Training two-way shape memory alloy by reheat treatment,” J. Materials Sci. Letters 19:1549-1550 (2000), Huang et al., “Micro gripper using two-way NiTi shape memory alloy thin sheet,” Materials Science Forum 394-3:95-98 (2002), and U.S. Pat. No. 5,882,444, the disclosures of which are fully incorporated herein by reference.

With apparatus 200, in training the shape memory material to have a two-way shape memory the thermally responsive member 214 is processed to have a disengaged state at temperatures around normal body and below, and an engaged state at some temperature above body temperature. In one embodiment, for example, the thermally responsive member 214 has a transition temperature at which it changes state of at least about 38° C. In FIG. 2, the thermally responsive member 214 is shown in both an engaged state 214(a) and disengaged state 214(b). Although the illustrated embodiment engages the coupler 208 by expansion and releases the coupler 208 by contraction, it will be appreciated that in an alternative embodiment, the thermally responsive member 214 may be trained in the opposite manner, i.e., to engage the coupler 208 by contraction and release the coupler 208 by expansion.

The thermally responsive member 214 has a proximal end that is in operable connection with, or in close proximity to, a resistive heat element 116, and energy is transferred to the thermally responsive member 214 from the resistive heat element 116 in a similar manner as has been previously described with the transfer of energy from the resistive heat element 116 to the thermally responsive member 114 of apparatus 100. The distal end of the thermally responsive member 214 is specially configured to engage the coupler 208, and specifically to engage an orifice 213 in the body wall of the opening 212 of the coupler 208. The distal end of the thermally responsive member 214 includes an elongate arm 215 that extends distally from the thermally responsive member 214. A detent 217 is disposed on the distal end of the elongate arm 215 and, when the distal end of the thermally responsive member 214 is placed within the opening 212 of the coupler 208, the detent 217 is oriented towards the body wall of the opening 212. The embodiment shown in FIG. 2 has a thermally responsive member 214 that incorporates a pair of elongate arms 215 and detents 217, with the detents 217 being opposed from each other and aligned to engage different orifices 213 when properly placed within the opening 212 of the coupler 208.

In the illustrated embodiment, to engage the implant device 104 to the pusher member 102 the distal end of the thermally responsive member 214 is placed within the opening 212 of the coupler 208 and oriented such that the detents 217 are aligned with the orifices 213 in the body wall of the opening 212. Energy from the resistive heat element 116 is applied to the thermally responsive member 214, thereby causing the thermally responsive member 214 to transition to an engaged state 214(a). In the engaged state 214(a), the detents 217 of the thermally responsive member 214 engage the orifices 213 in the body wall of the opening 212 of the coupler 208. The application of energy to the thermally responsive member 214 is maintained during the period in which it is desired to keep the member 214 in the engaged state 214(a), i.e., when it is desired to engage the implant device 104 with the pusher member 102.

To detach the implant device 104 from the pusher member 102, such as, e.g., after placing the implant device 104 at a desired location at or near a target cavity site, the application of energy from the resistive heating element 116 is discontinued. The temperature of the thermally responsive member 214 then decreases due to the relatively cooler ambient temperature, i.e., the surrounding body and blood temperature is cooler than the temperature required to place the thermally responsive member 214 in the engaged state 214(a). A biocompatible fluid, such as, e.g., saline, may optionally be manually applied through the pusher member 102 to accelerate the cooling process. When the thermally responsive member 214 cools to a sufficiently low temperature relative to the temperature required for the engaged state 214(a), the thermally responsive member 214 transitions to a disengaged state 214(b) in which the detents 217 retract from the orifices 213, thereby releasing the implant device 104 from the pusher member 102. The pusher member 202 may then be withdrawn proximally, leaving the implant device 104 at a desired target cavity site.

In an alternative embodiment, the thermally responsive member 214 may include a single elongate arm 215 and detent 217 (rather than the pair of elongate arms 215 and detents 217 shown in the embodiment illustrated in FIG. 2) or, in a further alternative, may incorporate a plurality of elongate arms 215 and corresponding detents 217. Regardless of the number of elongate arms 215 and detents 217, the coupler 208 will have at least as many orifices 213 in the body wall of the opening 212 as there are detents 217.

In addition to the illustrated embodiment, the thermally responsive member 214 may take a number of other mechanical configurations. For example, U.S. Pat. No. 6,102,917, U.S. Pat. No. 6,099,546, U.S. Pat. No. 5,645,564, U.S. Pat. No. 5,601,600, and U.S. Pat. No. 5,217,484 all describe mechanical detachment systems that could be used as part of the thermally responsive member 214. The disclosures of these patents are fully incorporated herein by reference.

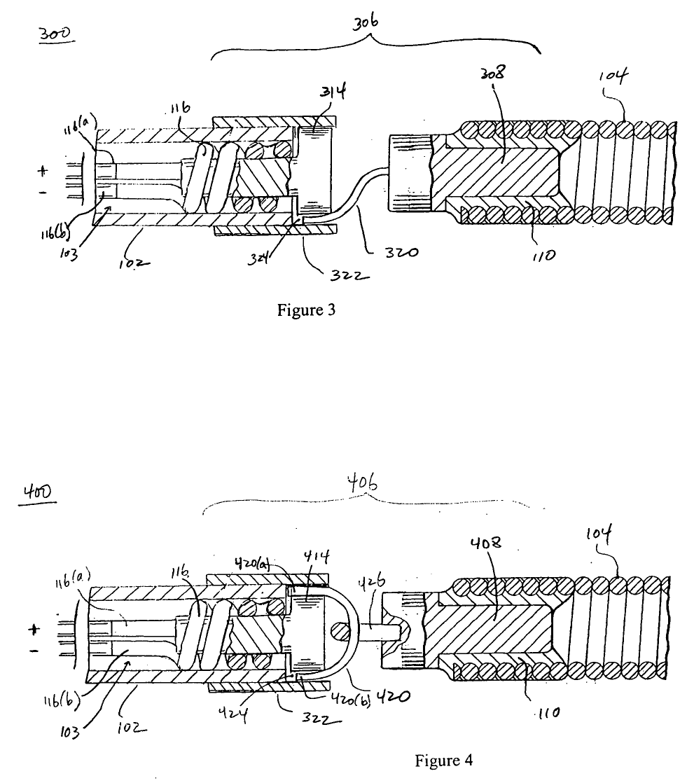

Turning to FIG. 3, a further embodiment of the present invention, implant device delivery apparatus 300, is shown. As with apparatus 200, apparatus 300 shares several common elements with apparatus 100. For example, the devices usable as the implant device 104 with apparatus 100 and 200 are also usable as the implant device 104 with apparatus 300, including, e.g., an embolic microcoil/coil, a continuous filamentous extrusion of polymeric “transition material,” a flexible filamentous carrier having one or more expansible, hydrophilic embolizing elements, a flexible filamentous carrier having a hydrophilic element at its distal end, a stent, a vascular filter, and the like. These implant devices 104 have been previously described with respect to apparatus 100. In addition to implant device 104, the same identification numbers are used to identify other elements/components of apparatus 100 that are also used in apparatus 300. Reference is made to the description of these elements in the description of apparatus 100 as that description also applies to these elements in apparatus 300.

Apparatus 300 includes a coupling section 306 used to engage and disengage the implant device 104 from the pusher member 102 through the use of a tether 320 that is selectively engaged by a thermally responsive member 314. The thermally responsive member 314 includes a proximal end that is surrounded by or in close proximity to a resistive heating element 116 in substantially the same manner as the thermally responsive members 114 and 214 of apparatus 100 and 200, respectively. The distal end of the thermally responsive member 314 preferably extends distally from the distal end of the pusher member 102. The diameter of the distal end of the thermally responsive member 314 is substantially the same as, or marginally larger than, the diameter of the distal end of the pusher member 102, whereas the remaining portions of the thermally responsive member 314, include the proximal end thereof, have a diameter sized to fit within the lumen 103 of the pusher member 102. Accordingly, the distal end of the thermally responsive member 314 is disposed outside of the distal end of the pusher member 102, and does not fit within the lumen 103 of the pusher member 102.

The coupling section 306 further includes a tubular retaining sleeve 322. In a preferred embodiment, the retaining sleeve 322 resists radial expansion. The retaining sleeve 322 may also be formed from a biocompatible metal or a combination of a biocompatible metal and a biocompatible polymeric material. Preferably, the retaining sleeve 322 is formed in part of a polymeric material such as polyethylene, polyethylene terephthalate (“PET”), polyamide, polyimide, polyester, polyurethane, or other suitable biocompatible material. If the retaining sleeve 322 is formed at least in part from a polymeric material, the material preferably has a durometer (Shore hardness) of at least 30D in order to provide an increased resistance to radial expansion. The retaining sleeve 322 surrounds at least the distal end of the thermally responsive member 314. In the embodiment shown in FIG. 3, the retaining sleeve 322 surrounds the distal end of the pusher member 202 as well as a part of the distal end of the thermally responsive member 314. The interior wall of the retaining sleeve 322 is permanently affixed to at least a portion of the outer circumference of the distal end of the thermally responsive member 314, but is not permanently affixed to the entire outer circumference thereof. As a result, at least one gap 324 exists between a portion of the interior wall of the retaining sleeve 322 and a portion of the outer circumference of the distal end of the thermally responsive member 314. The gap 324 is sufficiently large such that at least a portion of the proximal end of the tether 320 may be inserted into the gap 324.

The tether 320 may be formed using any biocompatible or implant material known in the art including, e.g., various biocompatible polymers, metals, biological materials, combinations thereof, and the like. Preferably, the tether 320 is elastic. In the embodiment shown in FIG. 3, the tether 320 is fixedly attached at its distal end to the proximal end of a coupler 308. At least the distal end of the coupler 308 is affixed to the distal portion of the implant device 104 in the same manner as the couplers 108 and 208 are affixed to the implant device 104, e.g., through the use of a suitable bond or weld 110, as previously disclosed. The unattached proximal end of the tether 320 is freely insertable into the gap 324.

The thermally responsive member 314 is similar to the thermally responsive member 114 of apparatus 100 in that the thermally responsive member 314 expands due to thermal expansion when energy is applied to it via the resistive heating element 116. As with the thermally responsive member 114, the thermally responsive member 314 is preferably formed from a material that expands to a sufficient diameter when the member 314 reaches a temperature of at least about 38° C. Accordingly, the thermally responsive member 314 is preferably made of a material with a relatively high coefficient of thermal expansion, such as, e.g., a material having a coefficient of thermal expansion of at least about 1.5×10⁻⁵ mm/mm/° C. Suitable materials include a metal, a polymer, or a composite material so long as the material has a sufficiently high coefficient of thermal expansion.

By applying energy to the thermally responsive member 314, in this case from the resistive heating element 116, the thermally responsive member 314 is heated to a temperature sufficient to increase the diameter of the thermally responsive member 314, and specifically the distal end thereof, such that the distal end of the thermally responsive member 314 narrows the gap 324 between the outer circumference of the thermally responsive member 314 and the interior wall of the retaining sleeve 322. When the gap 324 is narrowed via the application of energy to the thermally responsive member 314, the portion of the proximal end of the tether 320 inserted into the gap 324 is frictionally engaged in the gap 324 between the outer circumference of the member 314 and the interior wall of the retaining sleeve 322. In this configuration, the implant device 104 is engaged to the pusher member 102, energy is continually applied to the thermally responsive member 314 to maintain the engaged state (and keep the implant device 104 engaged to the pusher member 102). Alternatively, energy is intermittently applied but with a sufficient frequency to maintain the thermally responsive member 314 in the enlarged state. The frictional engagement has a resistance that is sufficiently high such that an axial force is required to overcome the frictional engagement and thereby detach the implant device 104 from the pusher member 102. In one embodiment, the axial force required to detach the implant device 104 from the pusher member 102 when the proximal end of the tether 320 is frictionally engaged in the gap 324 is at least about 45 grams (0.1 lbs). When the implant device 104 is secured to the pusher member 102 in this manner, the implant device 104 may be maneuvered within the body of a patient and placed at or near a target cavity site by manipulating the pusher member 102.

After the implant device 104 is maneuvered to or placed near the target cavity site, or when it is otherwise desired to disengage the implant device 104 from the pusher member 102, the application of energy to the thermally responsive member 314 is discontinued. The thermally responsive member 314 then contracts as it cools due to the relatively cooler ambient temperature, e.g., the relatively cooler body or blood temperature. As with apparatus 100, a biocompatible fluid such as saline may optionally be applied to the thermally responsive member 314 by injecting the fluid into the lumen 103 of the pusher member 102 in order to accelerate the cooling process. As the thermally responsive member 314 cools and contracts, the gap 324 widens because the outer circumference of the thermally responsive member 314 contracts away from the interior wall of the retaining sleeve 322. The tether 320 is then released from the gap 324, i.e., the thermally responsive member 314 and the retaining sleeve 322 no longer frictionally engage the tether 320 within the gap 324, and the implant device 104 may be disengaged from the pusher member 102. Accordingly, the pusher member 102 may be withdrawn proximally from the body, leaving the implant device 104 at the desired target cavity site.

FIG. 4 illustrates another embodiment of the present invention, implant device delivery apparatus 400. Like apparatus 300, apparatus 400 shares common elements with the other embodiments previously disclosed, including apparatus 100 and 200, as well as apparatus 300 (e.g., the retaining sleeve 322). The common elements are designated by the same identification numbers in FIG. 4 that have previously been used in describing those comment elements with respect to apparatus 100, 200, and 300. Reference is also made to the previous descriptions of these elements as those descriptions also apply to the same elements in apparatus 400. The implant device 104 of apparatus 400, for example, is the same as the implant device 104 in apparatus 100, 200, and 300 in that the implant device 104 may be an embolic microcoil/coil, a continuous filamentous extrusion of polymeric “transition material,” a flexible filamentous carrier having one or more expansible, hydrophilic embolizing elements, a flexible filamentous carrier having a hydrophilic element at its distal end, a stent, a vascular filter, and the like.

Apparatus 400 incorporates a coupling section 406 that, in a manner similar to the coupling section 306 of apparatus 300, is used to selectively engage the implant device 104 with the pusher member 202 by using a tether 420. The coupling section 406 includes a thermally responsive member 414. The proximal end of the thermally responsive member 414, as with the thermally responsive members of the other embodiments of the present invention, is surrounded by or in close proximity to a resistive heating element 116 and also lies within the lumen 103 of the pusher member 102. The distal end of the thermally responsive member 414 preferably extends distally from the distal end of the pusher member 102. The thermally responsive member 414 has a distal end that is preferably larger in diameter than at least the proximal end of the member 414. The distal end of the thermally responsive member 414 is sized such that the tether 420 may be situated between the thermally responsive member 414 and a retaining sleeve 322. Similar to thermally responsive members 114 and 314, the thermally responsive member 414 is preferably formed from a material that expands to a desired diameter when energy is applied to the member, such as, e.g., when sufficient energy is applied to the member 414 for the member 414 to reach a temperature of at least about 38° C. Therefore, the thermally responsive member 414 is preferably made of a material with a relatively high coefficient of thermal expansion, e.g., a coefficient of thermal expansion of at least about 1.5×10⁻⁵ mm/mm/° C. Exemplary materials include a metal, a polymer, or a composite material with a sufficiently high coefficient of thermal expansion. Optionally, the thermally responsive member 414 may be coated with a biocompatible material covering, such as, e.g., polytetrafluoroethylene or PTFE, to improve blood contact biocompatibility and/or thermal insulation.

The tether 420 of the coupling section 406 is preferably elastic and formed using any biocompatible or implant material known in the art including, e.g., various biocompatible polymers, metals, biological materials, combinations thereof, and the like. The tether 420 has a first end 420(a) and a second end 420(b). The first end 420(a) of the tether 420 is permanently affixed in a space between the distal end of the thermally responsive member 414 and the retaining sleeve 322. The second end 420(b) of the tether 420 is not permanently affixed to the thermally responsive member 414 or the retaining sleeve 322 but, instead, may be selectively engaged in a gap 424 between the outer circumference of the distal end of the thermally responsive member 414 and the interior wall of the retaining sleeve 322.

The coupling section 406 also incorporates a coupler 408. At least the distal end of the coupler 408 is affixed to the distal portion of the implant device 104 using, e.g., a bond or weld 110. As illustrated in FIG. 4, the distal end of the coupler 408 is preferably affixed within the interior space of the implant device 104. The coupler 408 incorporates an attachment point 426 protruding proximally from its proximal end and configured such that the body of the tether 420 may be placed therethrough. The attachment point 426 may be, e.g., formed in the shape of a ring or semicircle.

When the apparatus 400 is in an engaged state, i.e., the implant device 104 is engaged to the pusher member 102, the second end 420(b) of the tether 420 is engaged in the gap 424 by the thermally responsive member 414 and the retaining sleeve 322, as shown in FIG. 4. Additionally, the body of the tether 420 is looped through the attachment point 426 on the proximal end of the coupler 408, thereby securing the implant device 104 to the pusher member 102. When the apparatus 400 is in a disengaged state, the second end 420(b) of the tether 420 is freed from the gap 424, and the body of the tether 420 is no longer looped through the attachment point 426 of the coupler 408. Accordingly, the implant device 104 becomes removable from the pusher member 102.

To operate the apparatus 400, the apparatus 400 is first placed into the engaged state. The apparatus 400 is placed into the engaged state by looping the body of the tether 420 through the attachment point 426 of the coupler 408, and then placing the second end 420(b) of the tether 420 within the gap 424 between the distal end of the thermally responsive member 414 and the interior wall of the retaining sleeve 322. Energy is then applied to the thermally responsive member 414. In the illustrated embodiment, the resistive heating element 116 is used to apply heat energy to the thermally responsive member 414. As the energy is applied, the distal end of the thermally responsive member 414 expands and narrows the gap 424 until the second end 420(b) of the tether 420 is frictionally engaged between the outer circumference of the thermally responsive member 414 and the interior wall of the retaining sleeve 322. At this point, the apparatus 400 is in the engaged state, i.e., the implant device 104 is affixed to the pusher member 102. As with apparatus 100 and 300, the frictional engagement has a resistance that is sufficiently high such that an axial force is required to overcome the frictional engagement and thereby detach the implant device 104 from the pusher member 102. In one embodiment, the axial force required to detach the implant device 104 from the pusher member 102 when the second end 420(b) of the tether 420 is frictionally engaged in the gap 424 is at least about 45 grams (0.1 lbs). In one embodiment, energy is continually applied to the thermally responsive member 414 in order to maintain the apparatus 400 in the engaged state. In another embodiment, energy is intermittently applied but with a sufficient frequency to maintain the engaged state. While in the engaged state, the implant device 104 may be maneuvered to or near a desired target cavity site by using the pusher member 102 to position the implant device 104.

When it is desired to deliver the implant device 104, and therefore to disengage the implant device 104 from the pusher member 102, the application of energy to the thermally responsive member 414 is stopped. When the application of energy is discontinued, the thermally responsive member 414, i.e., the distal end of the thermally responsive member 414, cools and contracts since it is being exposed to a relatively cooler body or blood temperature. Furthermore, a biocompatible fluid such as saline may optionally be placed within the lumen 103 of the pusher member 102 and delivered to the thermally responsive member 414 to accelerate the cooling process. As a result of the contraction of the distal end of the thermally responsive member 414, the gap 424 widens and the second end 420(b) of the tether 420 is no longer frictionally engaged between the outer circumference of the thermally responsive member 414 and the interior wall of the retaining sleeve 322. The pusher member 102 may then be pulled back proximally away from the implant device 104, and the implant device 104 becomes disengaged from the pusher member 102 and left at the desired target cavity site.

In an alternative embodiment of the present invention, an implant device delivery apparatus similar to apparatus 300 and 400 is provided. With this alternative embodiment, when the apparatus is in an engaged state, a tether is frictionally engaged between two thermally responsive members or within an opening or slot in the distal end of a single thermally responsive member, rather than being engaged in a gap between the distal end of the thermally responsive member and the retaining sleeve. Other than this difference, this alternative apparatus is substantially similar to, and may be used in substantially the same manner, as apparatus 300 and 400.

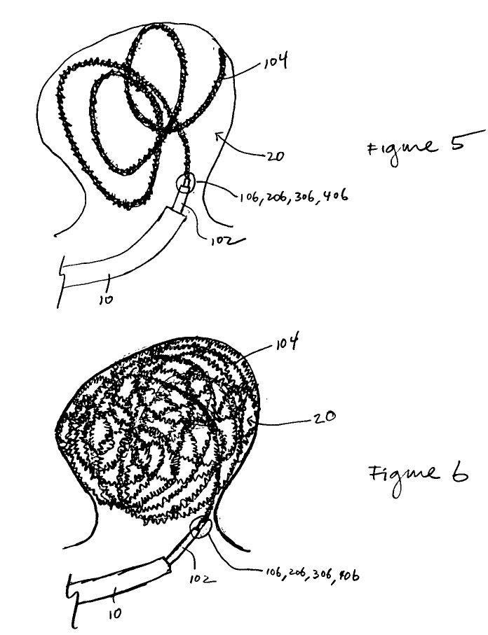

The present invention also provides for methods of delivering and releasing an implant device 104 using any of the apparatuses 100, 200, 300, and 400 of the present invention, or any other suitable implant device delivery apparatus. Turning to FIG. 5 and FIG. 6, in one aspect of this method, an elongate tubular access device 10, such as, e.g., a cannula or a catheter is inserted into the body of a patient. The access device 10 is maneuvered in the body and placed in close proximity to a desired target cavity site 20, which may be sites within blood vessels and vascular sites, such as, e.g., aneurysms and fistula, heart openings and defects, such as, e.g., the left atrial appendage, and other luminal organs, such as, e.g., fallopian tubes. One of the apparatuses 100, 200, 300, and 400 of the present invention is next prepared for use by placing the apparatus 100, 200, 300, 400 into an engaged state in which the implant device 104 is secured to a pusher member 102. This is accomplished, as previously discussed herein, by applying energy to a thermally responsive member 114, 214, 314, 414 located at the distal end of the pusher member 102. When energy is applied, the thermally responsive member 114, 214, 314, 414 engages a coupler 108, 208, 308, 408 affixed to the implant device 104. FIG. 5 and FIG. 6 specifically show the coupling section 106, 206, 306, 406 of the apparatus 100, 200, 300, 400. As previously described, both the thermally responsive member 114, 214, 314, 414 and the coupler 108, 208, 308, 408 form part of the coupling section 106, 206, 306, 406. The application of energy is maintained in order to keep the thermally responsive member 114, 214, 314, 414 engaged and secured to the coupler 108, 208, 308, 408, thereby keeping the implant device 104 secured to the pusher member 102. The application of energy may be continuous or, alternatively, intermittent with a sufficient frequency to keep the implant device 104 secured to the pusher member 102. In one aspect of this method, energy is applied to the apparatus 100, 200, 300, 400 prior to a physician or other user removing the apparatus 100, 200, 300, 400 from a product package. Here, the product package for the apparatus 100, 200, 300, 400 includes the apparatus 100, 200, 300, 400 already configured to be placed into the engaged state but for the application of energy. As result, potential problems that may arise from the inability of the user to correctly engage the implant device 104 and the pusher member 102 prior to the application of energy and placement of the apparatus 100, 200, 300, 400 in the engaged state are avoided.

After the apparatus 100, 200, 300, 400 is placed in and maintained in the engaged state, the distal end of the apparatus 100, 200, 300, 400 on which the implant device 104 is located is inserted coaxially into the lumen of the access device 10. Prior to the insertion of the apparatus 100, 200, 300, 400 into the lumen of the access device 10, a warm biocompatible solution, such as, e.g., saline, may optionally be injected into the lumen of the access device 10 to purge the access device 10. Purging the access device 10 with a warmed biocompatible solution prior to the insertion of the apparatus 100, 200, 300, 400 assists in preventing a premature cooling of the apparatus 100, 200, 300, 400 and, accordingly, helps prevent a premature or undesired transition of the apparatus 100, 200, 300, 400 from the engaged state to the disengaged state. When the optional purge step is performed, the biocompatible solution is preferably warmed to a temperature of at least 35° C. Irrespective of whether the purge step is performed, after the apparatus 100, 200, 300, 400 is inserted into the access device 10 the user advances the apparatus 100, 200, 300, 400 distally within the access device 10 and to the target cavity site 20. By doing so, the user positions the implant device 104 at the desired target cavity site 20 for deployment of the implant device 104. While the apparatus 100, 200, 300, 400 is being maneuvered within the access device 10, a warmed biocompatible solution, such as, e.g., saline warmed to at least 35° C., may optionally be injected into the lumen of the access device 10 to prevent a premature or undesired transition of the apparatus 100, 200, 300, 400 to the disengaged state. In an alternative aspect of this method, an access device 10 is not used and the user advances the apparatus 100, 200, 300, 400 directly within the body.

After the implant device 104 is placed in or near the desired target cavity site 20, and the site 20 is filled by the implant device 104 such that the device 104 forms a mass occupying a substantial portion of the target cavity site 20, the application of energy to the apparatus 100, 200, 300, 400 is discontinued, thereby removing the energy that had been applied to the thermally responsive member 114, 214, 314, 414 of the apparatus 100, 200, 300, 400. The thermally responsive member 114, 214, 314, 414 then cools, which results in the apparatus 100, 200, 300, 400 transitioning to a disengaged state. Specifically, the thermally responsive member 114, 214, 314, 414 and the coupler 108, 208, 308, 408 affixed to the implant device 104 separate, thereby allowing the pusher member 102 and the implant device 104 to be disengaged and separated from each other. To hasten the cooling process of the thermally responsive member 114, 214, 314, 414, the lumen 103 of the pusher member 102 may be filled with a biocompatible solution such as saline. The implant device 104 is left in or near the target cavity site 20, as desired, and the pusher member 102 is withdrawn proximally through the access device 10 (where the access device is used with the apparatus 100, 200, 300, 400) and out of the body. The access device 10 is then also withdrawn proximally out of the body. Additional, apparatus-specific details of methods for using the present invention have already been discussed herein in the detailed descriptions of each apparatus 100, 200, 300, 400.

In another aspect of this method, particularly when the implant device 104 is a microcoil/coil, implant devices 104 having coils of decreasing diameter may be sequentially delivered into the target cavity site 20 using the apparatus 100, 200, 300, 400. The sequential delivery of implant devices 104 of decreasing coil diameter is performed until the target cavity site 20 is sufficiently filled with implant devices 104. This aspect of the method may be performed when the size of the target cavity site 20 requires the use of more than one implant device 104.

For any of the embodiments of the present invention, the detachment of the implant device 104 may be detected using a separate electrical circuit or other methods known in the art, include the methods described in U.S. Pat. No. 6,397,850 and U.S. Pat. No. 5,643,254, the disclosures of which are fully incorporated herein by reference.

Though the invention has been described with respect to specific preferred embodiments, many variations and modifications will become apparent to those skilled in the art. It is therefore intended and expected that the appended claims be interpreted as broadly as possible in view of the prior art in order to include all such variations and modifications.

1. An implant device delivery and release apparatus comprising:
an implant device;
a pusher member having a proximal end and a distal end; and
a coupling section configured to attach the implant device to the distal end of the pusher member, the coupling section comprising
a thermally responsive member, and
a coupler adopted to receive the thermally responsive member,
wherein the thermally responsive member is held by the coupler by an application of energy and is released from the coupler by discontinuing the application of energy.

2. The apparatus of claim 1, wherein the coupling section further comprises a resistive heating element in communication with the thermally responsive member.

3. The apparatus of claim 1, wherein the pusher member further comprises a lumen, and the apparatus further comprises a resistive heating element disposed within the lumen of the pusher member and in communication with the thermally responsive member.

4. The apparatus of claim 1, wherein the coupling section further comprises a spring member disposed on distal end of the pusher member, the spring member being configured to forcibly release the thermally responsive member from the coupler after discontinuing the application of energy.

5. The apparatus of claim 1, wherein the coupler is in operable connection with the implant device, and the thermally responsive member is in operable connection with the pusher member.

6. The apparatus of claim 1, wherein the implant device comprises a hydrophilic element.

7. The apparatus of claim 1, wherein the implant device comprises a coil.

8. The apparatus of claim 1, wherein the thermally responsive member is released from the coupler upon cooling to body temperature.

9. A method for delivering an implant device using a pusher member having a proximal end and a distal end, the method comprising:
affixing the implant device to the distal end of the pusher member by applying energy to the pusher member;
introducing the implant device into a body having a target cavity;
advancing the implant device to the target cavity; and
detaching the implant device from the distal end of the pusher member by discontinuing the application of energy to the pusher member.

10. The method of claim 9, wherein affixing the implant device to the pusher member is performed prior to introducing the implant device into the body.

11. The method of claim 9, wherein affixing the implant device is performed using electrical energy.

12. The method of claim 9, wherein the implant device is a coil having a proximal end and a distal end, and affixing the implant device comprises coupling the proximal end of the coil to the distal end of the pusher member prior to applying energy to the pusher member.

13. The method of claim 9, further comprising removing the pusher member from the body after detaching the implant device from the pusher member.

14. An implant device delivery and release apparatus comprising:
an implant device;
a pusher member having a proximal end and a distal end; and
a coupling section configured to couple the implant device to the distal end of the pusher member, the coupling section comprising
a thermally responsive member,
a retaining sleeve surrounding the thermally responsive member, and
a tether detachably engaged between the thermally responsive member and the retaining element,
wherein the implant device is coupled to the pusher member by an application of energy to the coupling section and is released from the pusher member by discontinuing the application of energy.

15. The apparatus of claim 14, wherein the thermally responsive member has a first configuration in which the thermally responsive member engages the tether, and a second configuration in which the thermally responsive member disengages the tether.

16. The apparatus of claim 15, wherein the thermally responsive member is placed in the first configuration by an application of energy to the coupling section.

17. The apparatus of claim 14, wherein the coupling section further comprises a resistive heating element in communication with the thermally responsive member.

18. The apparatus of claim 14, wherein the tether is permanently attached to the implant device.

19. The apparatus of claim 14, wherein the tether comprises a first end and a second end, the first end being permanently engaged between the retaining sleeve and the thermally responsive member, and the second end being releasably engaged between the retaining sleeve and the thermally responsive member.

20. The apparatus of claim 19, wherein the second end of the tether is released from between the retaining sleeve and the thermally responsive member by discontinuing the application of energy.

21. The apparatus of claim 19, wherein the coupling section further comprises an attachment point to which the tether is detachably engaged, the tether being engaged to the attachment point by the application of energy and released from the attachment point by discontinuing the application of energy.

22. The apparatus of claim 21, wherein the implant device comprises a proximal end and a distal end, and the attachment point is disposed on the proximal end of the implant device.

23. The apparatus of claim 14, wherein the implant device comprises a coil.

24. The apparatus of claim 14, wherein the implant device comprises a coil and a hydrophilic element.
